(19) United States
(12) Patent Application Publication
(10) Pub. No.: US 2015/0345122 A1
(43) Pub. Date: Dec. 3, 2015

(54) TOILET FLUSH LEVER SANITIZING DEVICE

(71) Applicant: Rose Talbot, Port Saint Lucie, FL (US)

(72) Inventor: Rose Talbot, Port Saint Lucie, FL (US)

(21) Appl. No.: 14/693,214

(22) Filed: Apr. 22, 2015

Related U.S. Application Data
(60) Provisional application No. 62/003,680, filed on May 28, 2014.

(51) Int. Cl. E03D 9/00; A61L 2/24; A61L 2/22; E03D 5/092

(52) U.S. Cl. CPC E03D 9/002; E03D 5/092; A61L 2/24; A61L 2/22

(57) ABSTRACT

A toilet flush lever sanitizing device is provided. The device may include a housing to contain internal components. The present invention further includes a sensor operable to detect a motion of the toilet lever of the toilet. A pressurized sanitizer canister that contains a sanitizer is disposed within the housing. The canister includes a nozzle that is directed towards the toilet lever. When the sensor detects the motion of the toilet lever, the sanitizer canister delivers a spray of sanitizer from the nozzle after a period of time from the detection of the motion.

### CROSS-REFERENCE TO RELATED APPLICATION

This application claims the benefit of priority of U.S. provisional application No. 62/003,680, filed May 28, 2014, the contents of which are herein incorporated by reference.

### BACKGROUND OF THE INVENTION

The present invention relates to toilets and, more particularly, to a toilet flush lever sanitizing device.

A toilet is a sanitation fixture used primarily for the disposal of human urine and feces. They are often found in a small room referred to as a toilet, bathroom or lavatory. A toilet can be designed for people who prefer to sit (on a toilet pedestal) or for people who prefer to squat (over a squatting toilet). Flush toilets, which are common in many parts of the world may be connected to a nearby septic tank or more commonly in urban areas via sewer pipes connected to a sewerage pipe system. Typically, flush toilets include flush levers that are pulled to activate the flushing of the toilet. The flush levers are activated after a user has used the toilet. Therefore, the user's hands may be dirty and contain germs, which are in turn passed onto the flush lever.

As can be seen, there is a need for a device that sanitizes the flush lever.

### SUMMARY OF THE INVENTION

In one aspect of the present invention, a toilet flush lever sanitizing device comprises: a housing attachable to a toilet; a sensor operable to detect a motion of a toilet lever of the toilet; and a sanitizer canister comprising a nozzle directed towards the toilet lever, wherein when the sensor detects the motion of the toilet lever, the sanitizer canister delivers a spray of sanitizer from the nozzle after a period of time from the detection of the motion.

In another aspect of the present invention, a toilet flush lever sanitizing device comprises: a housing attachable to a toilet; a sanitizer canister comprising a nozzle directed towards the toilet lever; and an activator operably connected to the sanitizer canister, wherein when the activator is activated, the sanitizer canister delivers a spray of sanitizer from the nozzle.

These and other features, aspects and advantages of the present invention will become better understood with reference to the following drawings, description and claims.

### BRIEF DESCRIPTION OF THE DRAWINGS

FIG. 1 is a perspective view of an embodiment of the present invention in place on a toilet;

FIG. 2 is an exploded perspective view of an embodiment of the present invention in an open position;

FIG. 3 is a cross-sectional view of an embodiment of the present invention prior to being activated; and

FIG. 4 is a cross-sectional view of the embodiment of FIG. 3 showing the present invention in an activated mode.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

The present invention includes a unit secured to the top of toilet tank lid which automatically releases sanitizer on toilet lever after the toilet lever has been flushed. The next user will thereby not make contact with the prior person's germs or bodily fluids, eliminating cross contamination from people that had previously touched the toilet lever.

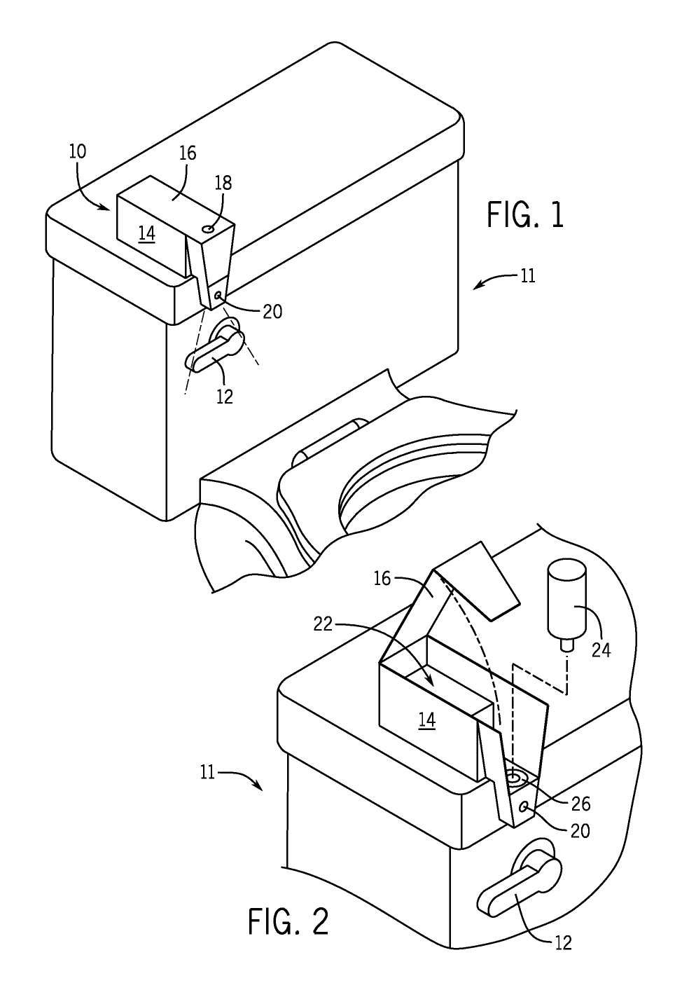

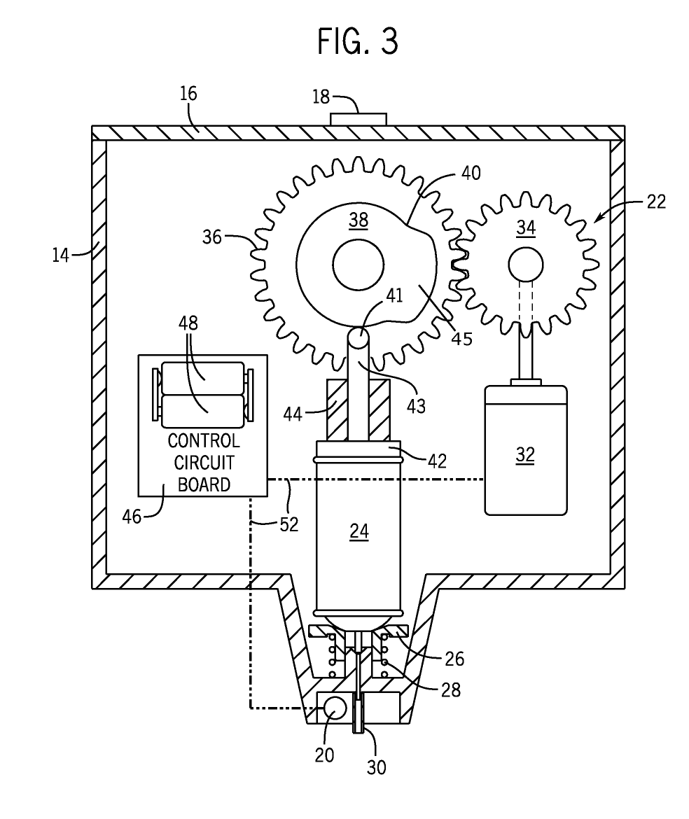

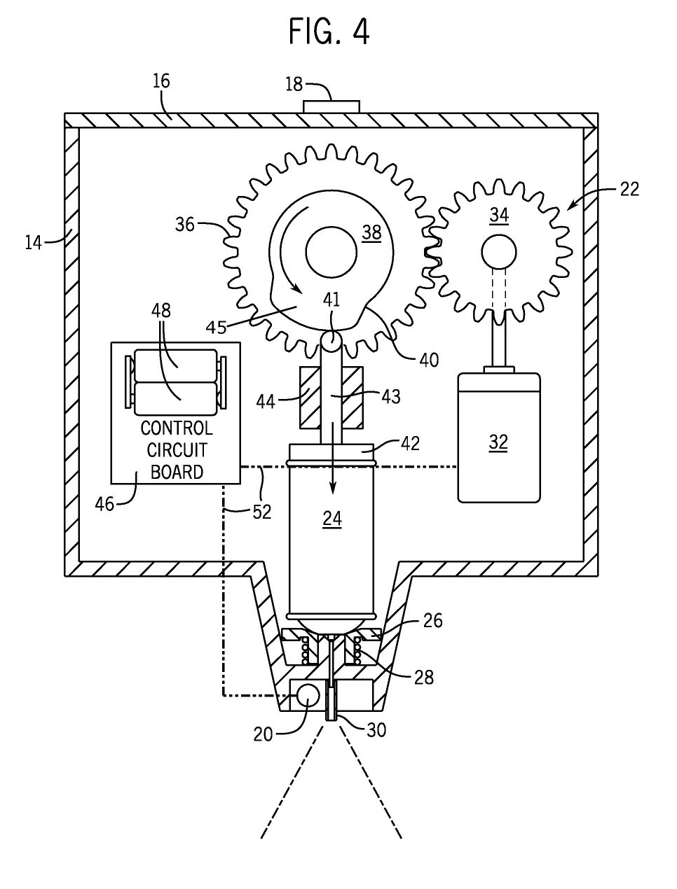

Referring to FIGS. 1 through 4, the present invention includes a toilet flush lever sanitizing device 10. The device 10 may include a housing 14 to contain internal components 22. The present invention further includes an activator to activate a sanitizer canister 24. The activator may be a sensor 20 operable to detect a motion of the toilet lever 12 of the toilet 11. The pressurized sanitizer canister 24 contains a sanitizer and is disposed within the housing 14. The canister 24 includes a nozzle 30 that is directed towards the toilet lever 12. When the sensor 20 detects the motion of the toilet lever 12, the sanitizer canister 24 delivers a spray of sanitizer from the nozzle 30 after a period of time from the detection of the motion.

The housing 14 of the present invention may be attached to the toilet lid above the toilet lever 12 so that the nozzle 30 is directed towards the lever 12. In certain embodiments, the housing 14 may include a lid 16 that may be removed to access the internal components 22. As illustrated in FIG. 2, the lid 16 may be hingedly connected to the housing 14 and may snap open and snap close. In certain embodiments, the lid 16 may be retained in the closed position by a lever. The lever may be activated by a release button 18 to open the lid 16. Opening the lid 16 allows a user access to the canister 24 so that the user may replace an empty canister 24 with a full canister 24. The lid 16 may also allow access to batteries 48, which may be replaced once depleted.

The sensor 20 of the present invention may include a motion sensor 20. However, the present invention may also include a pressure sensor built into the lever 12 or other sensor that indicates that the lever 12 has been pulled. The motion sensor 20 may be directed towards the lever 12 and detect motion of the user's hand as well as the lever 12 itself. Once the motion has been detected, the canister 24 may be activated after a delayed period of time to prevent the user's hand from being sprayed with sanitizer. In certain embodiments, the period of time may be about two seconds and up to about eight seconds, such as about three seconds up to about six seconds, such as about five seconds.

In certain embodiments, the present invention includes a drive motor 32. The drive motor 32 may be operable to activate the canister 24 so that the canister 24 may deliver the sanitizer. In such embodiments, the sensor 20 may be electrically connected to a circuit board 46, which in turn is electrically connected to the drive motor 32. The electrical components may be connected by electrical wire 52. When the sensor 20 detects that the lever 12 has been pulled, the sensor sends a signal to the circuit board 46, which in turn powers the drive motor 32.

As mentioned above, the present invention may include a delaying mechanism. For example, the present invention may include a first gear 34 and a second gear 36. The first gear 34 and the second gear 36 may interlock. A cam 38 may be attached to and rotated by the second gear 38. The cam 38 of the present invention may include a recessed portion 40 that tapers to a protruding portion 45. The present invention further includes a plunger 42 having a plunger shaft 43 and a follower pin 41 attached to the plunger shaft 43. The plunger shaft 43 may run through a guide 44 and the plunger 42 may rest against the canister 24. The canister 24 may be biased towards the plunger 42 by a spring 28 disposed about a canister trigger 26. In the deactivated position, the follower pin 41 may rest within the recessed portion 40. When the sensor 20 is activated, the driver motor 32 rotates the first gear 34, which in turn rotates the second gear 36. The cam 38 is then rotated so that the follower pin 40 slides about the tapered portion of the cam 38. The follower pin 40 rotates until engaging the protruding portion 45, thereby pushing the plunger 42 against the canister 24 and activating a pressure activator attached to the canister 24. While the follower pin 40 travels along the tapered portion, the period of time, such as about three to five seconds, elapses, and the sanitizer is deployed.

In alternate embodiments, the present invention may be run by a belt drive and may run off of the power grid or solar energy. The present invention could be manual and include a push button or a foot step peddles. The present invention could be activated by a remote or a voice control. The present invention could be reconfigured to hang above, on the side of or below the toilet.

It should be understood, of course, that the foregoing relates to exemplary embodiments of the invention and that modifications may be made without departing from the spirit and scope of the invention as set forth in the following claims.

What is claimed is:

1. A toilet flush lever sanitizing device comprising:
a housing attachable to a toilet;
a sensor operable to detect a motion of a toilet lever of the toilet; and
a sanitizer canister comprising a nozzle directed towards the toilet lever,
wherein when the sensor detects the motion of the toilet lever, the sanitizer canister delivers a spray of sanitizer from the nozzle after a period of time from the detection of the motion.

2. The toilet flush lever sanitizing device of claim 1, wherein the sensor is a motion sensor operable to detect a motion near the toilet lever.

3. The toilet flush lever sanitizing device of claim 1, wherein the period of time ranges from about two seconds up to about eight seconds.

4. The toilet flush lever sanitizing device of claim 1, further comprising a drive motor electrically connected to the sensor via a control circuit board, wherein the drive motor is operable to activate the sanitizer canister.

5. The toilet flush lever sanitizing device of claim 4, wherein the nozzle of the sanitizer canister comprises a pressure activator.

6. The toilet flush lever sanitizing device of claim 5, further comprising:
a first gear rotatable by the driver motor;
a second gear interlocked with the first gear;
a cam rotatable by the second gear; and
a plunger disposed to press against the sanitizer canister applying pressure to the pressure activator when the cam is rotated.

7. The toilet flush lever sanitizing device of claim 6, wherein the sanitizer canister is biased towards the cam by a spring suspended about a canister trigger, wherein the rotation of the cam presses the sanitizer canister against the canister trigger, thereby activating the sanitizer canister.

8. The toilet flush lever sanitizing device of claim 7, wherein the plunger comprises a plunger shaft and a follower pin attached to the plunger shaft.

9. The toilet flush lever sanitizing device of claim 8, wherein the cam comprises a recessed portion that tapers to a protruding portion, wherein the cam is rotated so that the follower pin travels from the recessed portion to the protruding portion, thereby applying pressure and activating the canister.

10. The toilet flush lever sanitizing device of claim 1, wherein the housing comprises a hinged lid operable to open and close to gain access to the sanitizer canister.

11. A toilet flush lever sanitizing device comprising:
a housing attachable to a toilet;
a sanitizer canister comprising a nozzle directed towards the toilet lever; and
an activator operably connected to the sanitizer canister,
wherein when the activator is activated, the sanitizer canister delivers a spray of sanitizer from the nozzle.

12. The toilet flush lever sanitizing device of claim 11, wherein the activator is a motion sensor operable to detect a motion near the toilet lever wherein when the sensor detects the motion of the toilet lever, the sanitizer canister delivers a spray of sanitizer from the nozzle after a period of time from the detection of the motion.

13. The toilet flush lever sanitizing device of claim 12, wherein the period of time ranges from about two seconds up to about eight seconds.

14. The toilet flush lever sanitizing device of claim 12, further comprising a drive motor electrically connected to the sensor via a control circuit board, wherein the drive motor is operable to activate the sanitizer canister.

15. The toilet flush lever sanitizing device of claim 14, wherein the nozzle of the sanitizer canister comprises a pressure activator.

16. The toilet flush lever sanitizing device of claim 15, further comprising:
a first gear rotatable by the driver motor;
a second gear interlocked with the first gear;
a cam rotatable by the second gear; and
a plunger disposed to press against the sanitizer canister applying pressure to the pressure activator when the cam is rotated.

17. The toilet flush lever sanitizing device of claim 16, wherein the sanitizer canister is biased towards the cam by a spring suspended about a canister trigger, wherein the rotation of the cam presses the sanitizer canister against the canister trigger, thereby activating the sanitizer canister.

18. The toilet flush lever sanitizing device of claim 17, wherein the plunger comprises a plunger shaft and a follower pin attached to the plunger shaft.

19. The toilet flush lever sanitizing device of claim 18, wherein the cam comprises a recessed portion that tapers to a protruding portion, wherein the cam is rotated so that the follower pin travels from the recessed portion to the protruding portion, thereby applying pressure and activating the canister.

20. The toilet flush lever sanitizing device of claim 11, wherein the housing comprises a hinged lid operable to open and close to gain access to the sanitizer canister.
